(19) United States
(12) Patent Application Publication
(10) Pub. No.: US 2016/0367831 A1
(43) Pub. Date: Dec. 22, 2016

(54) IMPLANTABLE MEDICAL DEVICE, SYSTEM AND METHOD FOR INDUCING THROMBOSIS IN AN ARTERY OF A LIVING ANIMAL

(71) Applicant: Chang Gung Memorial Hospital, Linkou, Taoyuan City (TW)

(72) Inventors: Tsong-Hai Lee, Taipei City (TW); Shyh-Liang Lou, Hsinchu City (TW); Jih-Chao Yeh, Taoyuan City (TW)

(21) Appl. No.: 14/742,071

(22) Filed: Jun. 17, 2015

(51) Int. Cl. A61N 5/06

(52) U.S. Cl. CPC A61N 5/062; A61N 2005/0602; A61N 2005/0626; A61N 2005/0651; A61N 2005/0632

(57) ABSTRACT

A method for inducing thrombosis in an artery of a living animal includes: implanting an implantable medical device into a living animal, the implantable medical device including a light-emitting element and a wireless power receiver; administrating a photo-sensitizing dye material into the living animal; and providing wirelessly a control signal, from an external power controller, to the wireless power receiver which transmits electrical power to the light-emitting element in response to the control signal to drive the light-emitting element to emit light for irradiating the artery of the living animal.

### BACKGROUND OF THE INVENTION

Field of the Invention

The present disclosure relates to an implantable medical device, more particularly to a system and method for inducing thrombosis in an artery of a living animal using the implantable medical device.

Background Information

With the growing proportions of elderly people, stroke has now become a major health issue and the leading cause of death worldwide. Thrombosis is a diffuse pathologic process that starts with endothelial dysfunction and clinically manifests as coronary artery disease, cerebrovascular disease and transient ischemic attack. Considering the is critical role of thrombosis formation in many vascular diseases, several animal models have been developed to study the underlying mechanism and therapeutic options. Recently, arterial thrombosis induced by photochemical injury has become a widely-used animal model. Such photochemical injury model is believed to be similar to the mechanism of human arterial thrombosis caused by reactive oxygen species.

Conventionally, laser has been adopted for inducing photochemical injury of the animal models for thrombosis formation. However, the light power of laser irradiation (e.g., 9 V/cm2) is too strong and causes severe endothelial injury and rapid accumulation of platelets at the vessel wall, leading to immediate thrombotic occlusion. Moreover, heat generated by laser may cause hyperthermic injury to the living animal. Furthermore, in order to provide electrical power for laser irradiation, such conventional method cannot avoid transcutaneous wiring and may increase the risk of infection.

### SUMMARY OF THE INVENTION

Therefore, an object of the disclosure is to provide a system and/or method that can alleviate at least one of the aforementioned drawbacks of the prior art.

According to one aspect of the present disclosure, a method for inducing thrombosis in an artery of a living animal may include: implanting an implantable medical device into a living animal, the implantable medical device including a light-emitting element and a wireless power receiver; administrating a photo-sensitizing dye material into the living animal; and providing wirelessly a control signal, from an external power controller, to the wireless power receiver which transmits electrical power to the light-emitting element in response to the control signal to drive the light-emitting element to emit light for irradiating the artery of the living animal.

According to another aspect of the present disclosure, a system for inducing thrombosis in an artery of a living animal may include an external power controller and an implantable medical device. The external power controller is operable to wirelessly is transmit a control signal. The implantable medical device is configured to be implanted in the living animal and includes a light-emitting element, and a wireless power receiver which is configured to wirelessly receive the control signal from the external power controller. The wireless power receiver is operable to transmit electrical power to the light-emitting element in response to the control signal for driving the light-emitting element to emit light for irradiating the artery of the living animal.

According to yet another aspect of the present disclosure, an implantable medical device is configured to be implanted in a living animal and is operable to induce thrombosis in an artery of the living animal. Such implantable medical device may include an irradiation module and an encapsulator. The irradiation module is configured to be secured to the artery and includes a light-emitting element, and a wireless power receiver that is configured to wirelessly receive a control signal from an external power controller and that is operable to transmit electrical power to the light-emitting element in response to the control signal for driving the light-emitting element to emit light for irradiating the artery of the living animal. The encapsulator is made of a bio-compatible material and hermetically encapsulates the irradiation module.

### BRIEF DESCRIPTION OF THE DRAWINGS

Other features and advantages of the disclosure will become apparent in the following detailed description of the embodiments with reference to the accompanying drawings, of which:

FIG. 1 is a schematic diagram of an exemplary embodiment according to the present disclosure, illustrating a system for inducing thrombosis in a living animal;

FIG. 2 is a schematic block diagram of the system of the exemplary embodiment;

FIG. 3 is a schematic diagram of the exemplary embodiment, illustrating an implantable medical device of the system;

FIG. 4 is a schematic perspective view of the exemplary embodiment, illustrating the implantable medical device being secured to an artery of the living animal;

FIG. 5 is a flow chart of the exemplary embodiment, illustrating a method for inducing thrombosis in the artery of the living animal using the system;

FIG. 6 illustrates ultrasound artery images of Examples 1 and 2 and Comparative Example 1;

FIG. 7 illustrates ultrasound artery images of Example 3 and Comparative Examples 2 to 4;

FIG. 8 illustrates ultrasound artery images of Comparative Examples 5 and 6;

FIG. 9 illustrates Magnetic Resonance Angiography results of Example 3 and Comparative Example 2; and

FIG. 10 illustrates histologic and immunohistochemical staining results of Example 3 and Comparative Example 2.

### DETAILED DESCRIPTION

Before the disclosure is described in greater detail, it should be noted that like elements are denoted by the same reference numerals throughout the disclosure.

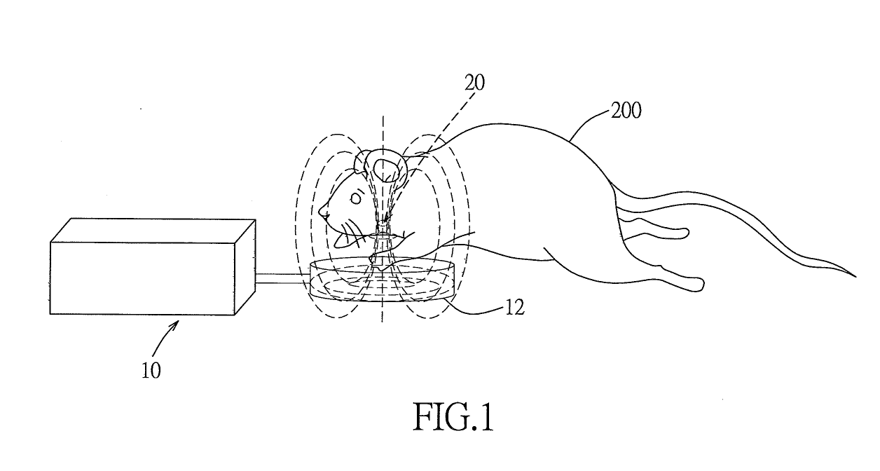

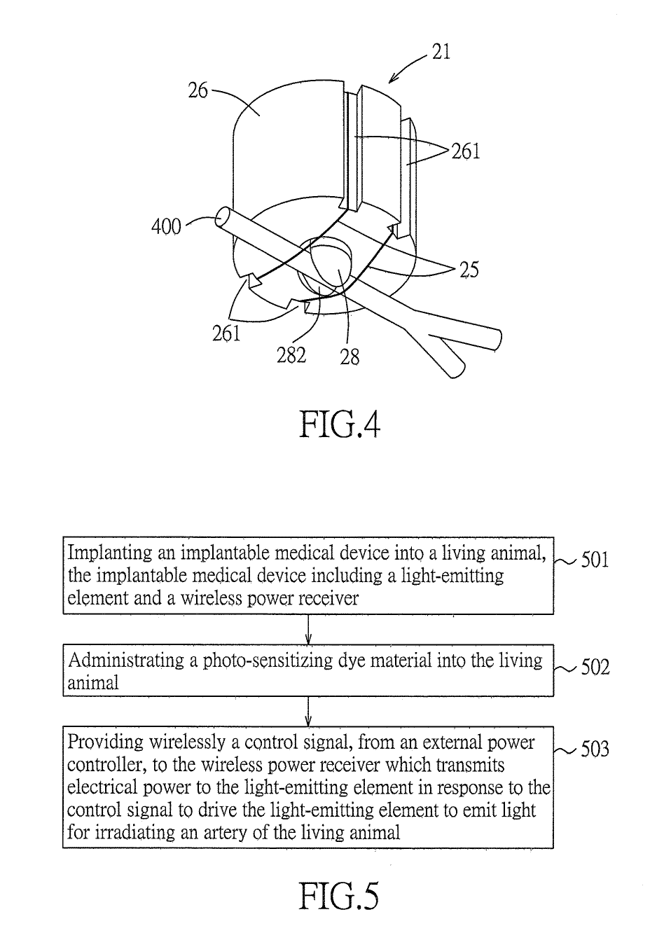

Referring to FIGS. 1 to 4, an exemplary embodiment of a system 100 for inducing thrombosis in an artery 400 of a living animal 200 (see FIG. 4) is shown to include an external power controller 10 and an implantable medical device 20.

It should be noted that, as exemplified in FIG. 1, the living animal 200 may be a rodent, such as a mouse or a rat (e.g., spontaneously hypertensive rats), but is not limited thereto according to the present disclosure. It should also be noted that the artery 400 may be, for example, a common carotid artery (CCA) in the living animal 200.

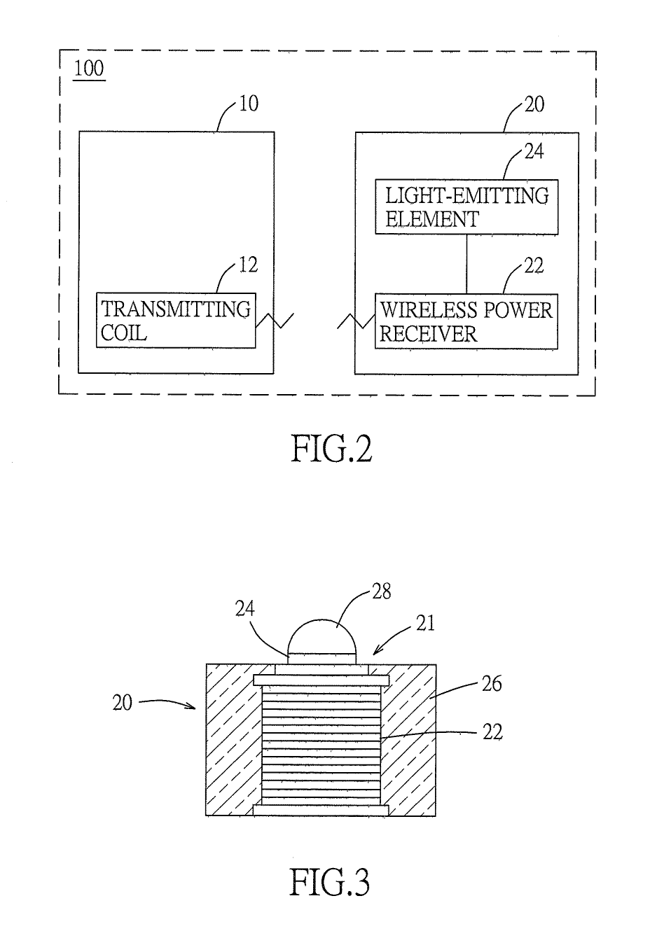

The external power controller 10 is operable to wirelessly transmit a control signal. As shown in FIG. 2, in this embodiment, the external power controller 10 includes an amplifier (such as a Class-E amplifier, not shown) and a transmitting coil 12 that is coupled to the amplifier and that is used to wirelessly transmit the control signal. The control signal may have a frequency of about 1 MHz, but is not limited thereto according to the present disclosure.

As shown in FIGS. 1 and 3, the implantable medical device 20 is configured to be implanted in the living animal 200 and includes an irradiation module 21 and an encapsulator 26.

The irradiation module 21 is configured to be secured to the artery 400 and includes a light-emitting element 24, and a wireless power receiver 22 that is configured to wirelessly receive the control signal from the transmitting coil 12 of the external power controller 10 and that is operable to transmit electrical power to the light-emitting element 24 in response to the control signal for driving the light-emitting element 24 to emit light for irradiating the artery 400 of the living animal 200. In this embodiment, the wireless power receiver 22 is configured as a coupling receiving coil which is resonant with the transmitting coil 12 of the external power controller 10. In this embodiment, the light-emitting element 24 is a surface-mount device type light-emitting diode (LED) that is configured to irradiate the artery 400 with a light power ranging from about 4.3 mW/cm2 to less than 15 mW/cm2. Use of the LED may reduce possible hyperthermic injury to the living animal 200. In certain embodiments, the light-emitting element 24 may have the light power of not less than about 6 mW/cm2. In certain embodiments, the light-emitting element 24 may have the light power ranging from 4.3 to 4.5 mW/cm2. As illustrated in FIGS. 3 and 4, the implantable medical device 20 may further include a lens element 28 disposed on the light-emitting element 24 for directing the light irradiated from the light-emitting element 24 onto the artery 40Q. The lens element 28 may be formed with a ditch 282 configured to receive the artery 400 as illustrated in FIG. 4, so that a targeted segment of the artery 400 can be effectively irradiated.

The encapsulator 26 is made of a bio-compatible material and hermetically encapsulates the irradiation module 21. The bio-compatible material 26 may be, but is not limited to, polydimethylsiloxane (PDMS). In this embodiment, as shown in FIG. 4, the encapsulator 26 is formed with at least one engaging groove 261 (four grooves are shown in FIG. 4), and the implantable medical device 2Q further includes at least one securing strap 25 that engages the engaging groove 261 and that is configured to secure the implantable medical device 20 to the artery 400.

Referring to FIG. 5, a method for inducing thrombosis in the artery 400 of the living animal 200 using the aforesaid system 100 according to the present disclosure is shown to include the following steps.

Step 501: implanting the implantable medical device 20 into the living animal 200.

Step 502: administrating a photo-sensitizing dye material into the living animal 20Q. The photo-sensitizing dye material can generate singlet oxygen radicals to damage the vascular endothelium cells upon light irradiation and initiate thrombus formation. In this embodiment, the photo-sensitizing dye material is rose bengal. The dose of the photo-sensitizing dye material for the living animal 200 may be, but is not limited to, 60 mg/kg.

Step 503: providing wirelessly the control signal, from the transmitting coil 12 of the external power controller 10, to the wireless power receiver 22 which transmits electrical power to the light-emitting element 24 in response to the control signal to drive the light-emitting element 24 to emit light for irradiating the artery 400 of the living animal 200. The light power of the light-emitting element 24 ranges from 4.3 mW/cm2 to 15 mW/cm2. The irradiated light from the light-emitting element 24 may have a wavelength (e.g., about 540 nm for rose bengal) to induce the photochemical reaction of the administrated photo-sensitizing dye material in the artery 400 of the living animal 200.

In some embodiments, the light-emitting element 24 is driven to irradiate light onto the artery 400 with a light power of not less than about 6 mW/cm2 continuously for a time period of about four hours. In such embodiments, the induced thrombosis in the artery 400 may be acutely formed. When the light power of the light-emitting element is lower than 5 mW/cm2 under the same time period of four hours, the induced thrombosis in the artery may gradually diminish and eventually disappear.

In some embodiments, the light-emitting element 24 may be driven to irradiate light onto the artery 400 with a light power that ranges from 4.3 to 4.5 mW/cm2 continuously for a first time period, then shut off after the first time period for a second time period, and further driven to irradiate the artery 40Q with a light power that ranges from 4.3 to 4.5 mW/cm2 continuously after the second time period for a third time period. In such embodiments, the first time period may be about two hours, the second is time period may be about thirty minutes, and the third time period may be about two hours. The induced thrombosis by the method of such embodiments can be progressively formed within a relatively long period, e.g., 7 days. When the light power of the light-emitting element 24 is less than 4.3 mW/cm2, the thrombosis may be formed transiently and may disappear within one day after irradiation. When the light power of the light-emitting element 24 is greater than 4.5 mW/cm2, the thrombosis may form but the artery 400 may become totally occluded one day after irradiation.

The method and system of the present disclosure are advantageous in various aspects:

1. The wireless power receiver 22 of the implantable medical device 20 according to the present disclosure allows the external power controller 10 to wirelessly provide electrical power to the light-emitting element 24 of the implantable medical device 20, so that transcutaneous wiring can be avoided and that risk of infection of the living animal 200 can be reduced. Moreover, the encapsulator 26 encapsulating the irradiation module 21 ensures bio-compatibility of the implantable medical device 20.

2. The light-emitting element 24 of the implantable medical device 20 can be regulated at various light powers to correspondingly induce acute or progressive thrombosis formation in the artery 40Q of the living animal 200.

3. The light power of the light-emitting element 24 according to the present disclosure is at a relatively low level in comparison to the conventional laser-induced method so as to avoid causing hyperthermic injury to the living animal 200.

The following examples are solely for the purpose of illustrating the exemplary embodiment and should not be construed as limiting the scope of the present disclosure.

### EXAMPLES

### Acute Thrombosis Occlusion

### Example 1

A system for inducing thrombosis was provided, including an implantable medical device and an external power controller. The external power controller includes a Class-E amplifier and a resonant transmitting coil. The implantable medical device includes a resonant receiving coil (i.e., the wireless power receiver) and an LED assembly (i.e., the irradiation module) including a surface-mount device type LED (i.e., the light-emitting element) and a LED lens (i.e., the lens element) disposed on the LED. The LED assembly and the resonant receiving coil were hermetically packaged with polydimethylsiloxane (PDMS) (i.e., the encapsulator). An open ditch of approximately 2 mm in width and depth was made on the LED lens, and four cuts (i.e., the engaging groove) were formed on the PDMS.

A male spontaneously hypertensive rat (SHR) (i.e., the living animal) was anesthetized with 3% isoflurane in a mixture of 30% oxygen and 70% nitrogen. After the administration of anesthesia, the rat was placed in a supine position. A 1 cm incision was made in the left inguinal area and the femoral vein was carefully dissected. A polyethylene tube to be used for rose bengal injection was inserted in the femoral vein via the dissected incision and secured using 3-0 nylon suture. Another procedure with a transverse incision in the inferior aspect of the sternal manubrium was made. Blunt dissection of the left common carotid artery (CCA) was performed. The CCA was placed into the ditch of the LED lens, and the implantable medical device was secured onto the CCA using 3-0 nylon suture (i.e., the securing strap) to engage the cuts on the PDMS.

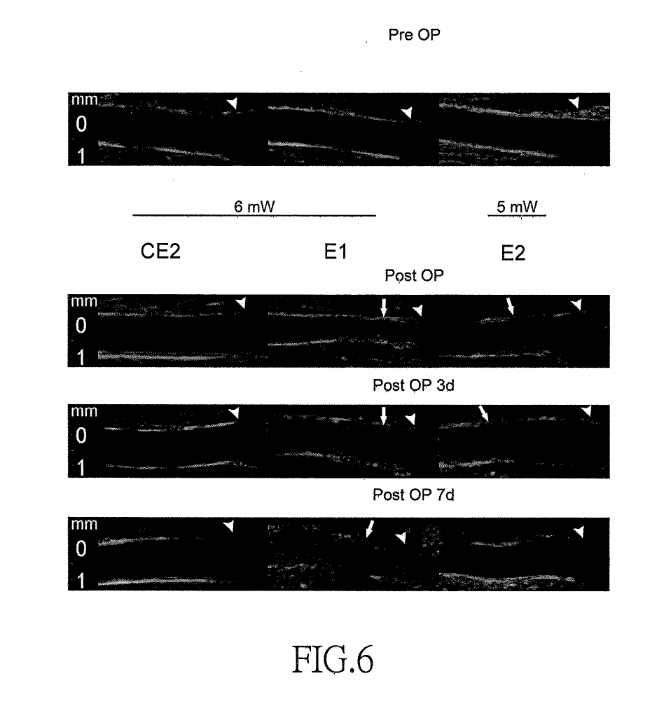

After the implantation procedure, the surgical wound was sutured and the rat was placed in a prone position near the transmitting coil of the external power controller. After isoflurane was decreased to 1%, rose bengal (Sigma-Aldrich, St. Louis, Mo., USA) was administrated intravenously at a dose of 60 mg/kg. Thereafter, the external power controller was powered to wirelessly drive the LED of the implantable medical device via the resonant receiving coil to irradiate the CCA at a wavelength of 540 nm. The LED irradiated light at a light power of 6 mW/cm2 continuously for 4 hours. The LED light power was measured using a Nova handheld laser power meter (Ophir Optronics Solutions, Ltd., Israel). After irradiation, the implantable medical device was removed and the wound was sutured using 3-0 nylon. Ultrasound images of the CCA prior to irradiation (Pre OP), immediately after irradiation (Post OP), 3 days after irradiation (Post OP 3d), and 7 days after irradiation (Post OP 7d) were captured and are shown in FIG. 6. It is clearly shown in FIG. 6 that the CCA of Example 1 was totally occluded right after irradiation and total occlusion was maintained for 7 days.

### Example 2

The method of Example 2 is similar to that of Example 1, with the only difference residing in that the LED light power was 5 mW/cm2. Ultrasound images for Example 2 prior to irradiation (Pre OP), immediately after irradiation (Post OP), 3 days after irradiation (Post OP 3d) and 7 days after irradiation (Post OP 7d) were captured and are shown in FIG. 6. Although the CCA of Example 2 was immediately occluded right after irradiation (Post OP) as indicated in FIG. 6, the formed thrombus gradually diminished 3 days after irradiation (Post OP 3d) and totally disappeared at 7 days after irradiation (Post OP 7d).

### Comparative Example 1

The difference between the methods of Example 1 and Comparative Example 1 resides in that no irradiation was performed in the method of Comparative Example 1. That is, the SHR of Comparative Example 1 was only administrated with rose bengal. Ultrasound images thereof prior to the administration of rose bengal (Pre OP), immediately after administrating the rose bengal (Post OP), 3 days after the administration (Post OP 3d) and 7 days after the administration (Post OP 7d) were captured and are illustrated in FIG. 6. The images show that thrombosis did not form in Comparative Example 1.

### Progressive Thrombosis Formation

### Example 3

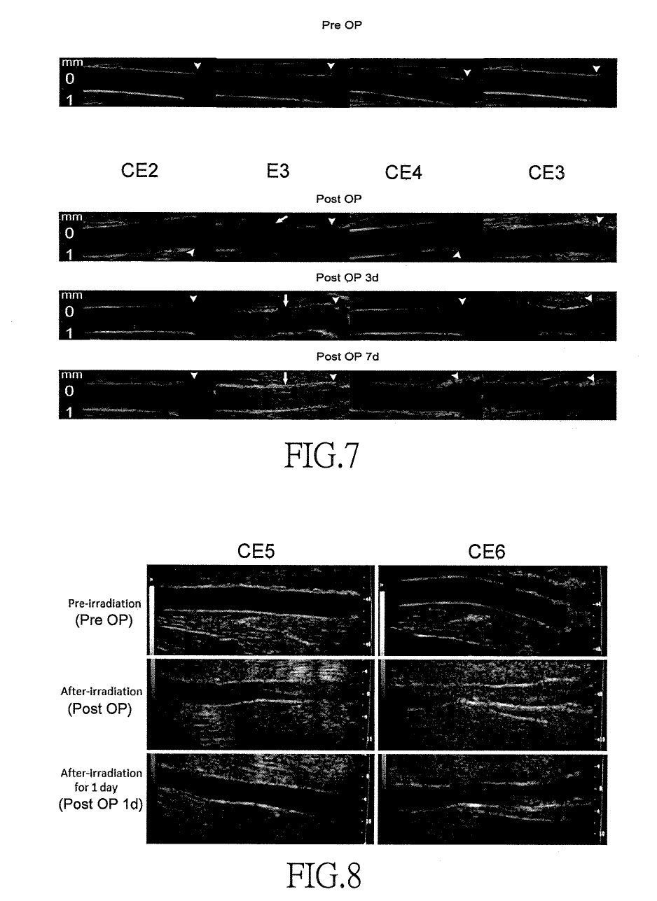

The method of Example 3 is similar to that of Example 1, with differences residing in the LED light power and the duration of irradiation. The LED of Example 3 irradiated the CCA at the light power of 4.5 mW/cm2 continuously for 2 hours, was subsequently turned off for 30 minutes, and then irradiated the CCA again at the light power of 4.5 mW/cm2 continuously for another 2 hours. Ultrasound images of the CCA prior to irradiation (pre OP), immediately after irradiation (Post OP), 3 days after irradiation (Post OP 3d) and 7 days after irradiation (Post OP 7d) were captured and are shown in FIG. 7. It is clearly shown that the induced thrombus gradually increased in is size and the CCA was totally occluded at 7 days after irradiation.

### Comparative Example 2

The difference between the methods of Example 3 and Comparative Example 2 resides in that the irradiation was omitted in the method of Comparative Example 2. That is, the CCA of Comparative Example 2 was only administrated with rose bengal (in fact, Example 3 and Comparative Example 2 respectively represent left and right CCAs of the same SHR). Ultrasound images of the CCA prior to irradiation in Example 3 (Pre OP), immediately after irradiation in Example 3 (Post OP), 3 days after irradiation in Example 3 (Post OP 3d) and 7 days after irradiation in Example 3 (Post OP 7d) were captured and are illustrated in FIG. 7. It is shown that thrombosis was not formed in the CCA of Comparative Example 2.

### Comparative Example 3

The method of Comparative Example 3 is similar to that of Example 3, with the only difference residing in that rose bengal was not administrated. Ultrasound images of the CCA prior to irradiation (Pre OP), immediately after irradiation (Post OP), 3 days after irradiation (Post OP 3d) and 7 days after irradiation (Post OP 7d) were captured and are illustrated in FIG. 7. It is shown that no thrombosis was formed in the CCA of Comparative Example 3.

### Comparative Example 4

The method of Comparative Example 4 was conducted without administration of rose bengal and without irradiation (in fact, Comparative Examples 3 and 4 respectively represent left and right CCAs of the same SHR). Ultrasound images of the CCA at various time points corresponding to Comparative Example 3 were captured and are illustrated in FIG. 7. It is shown that thrombosis was not formed in the CCA of Comparative Example 4.

### Comparative Example 5

The method of Comparative Example 5 was similar to that of Example 3, with the only difference residing in that the LED light power during the first and third time periods was at 4.0 mW/cm2. Ultrasound images of the CCA prior to irradiation (pre OP), immediately after irradiation (Post OP), and 1 day after irradiation (Post QP 1d) were captured and are shown in FIG. 8. It is shown that the thrombosis was formed transiently in the CCA of Comparative Example 5 and disappeared within one day after irradiation.

### Comparative Example 6

The method of Comparative Example 6 was similar to that of Example 3, with the only difference residing in that the LED light power during the first and third time periods was at 5.0 mW/cm2. Ultrasound images of the CCA prior to irradiation (pre OP), immediately after irradiation (Post OP), and 1 day after irradiation (Post OP 1d) were captured and are shown in FIG. 8. It is shown that although the thrombosis was formed in the CCA of Comparative Example 6, the artery became totally occluded one day after irradiation.

### [Magnetic Resonance Angiography (MRA) Examination]

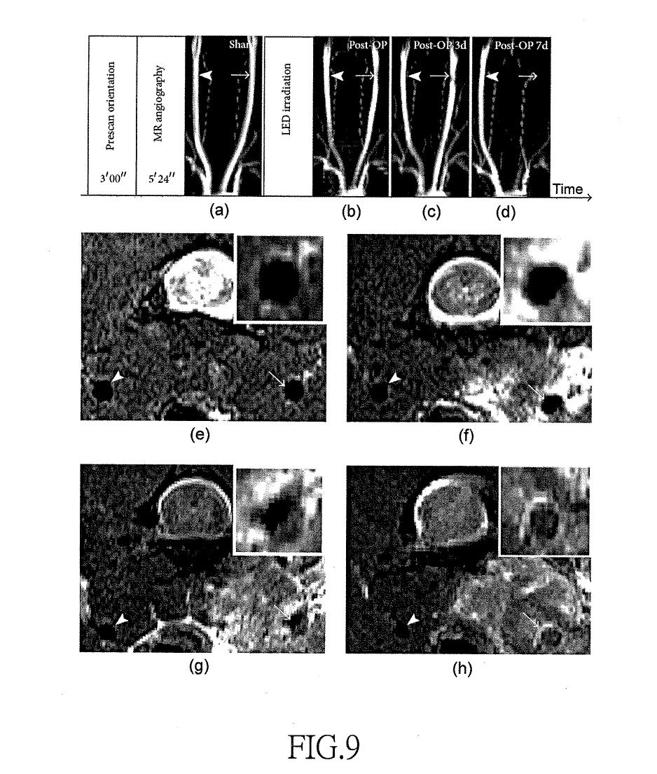

Serial in vivo MRA examination was performed on the CCAs of Example 3 and Comparative Example 2, and the typical MR imaging (MRI) experimental scheme of the time sequence is shown in FIG. 9. Images were acquired using a 7T MRI system (ClinScan 70/30 USR) (Bruker, Rheinstetten, Germany) with a linear body coil. The rat was anaesthetized with 1.5% isoflurane and connected to a respiratory rate monitor. The flow of anesthetic gas was constantly regulated to maintain the breathing rate at approximately 40/min. MRA of the CCAs was performed using the time-of-flight (TOF) method without a contrast agent. Transverse slices (panels (a) to (d) of FIG. 9) were acquired with a fast, low-angle shot (FLASH) using the following parameters: TR=22 ms, TE=4.87 ms, pulse angle=90 degrees, field of view (FQV)=55×42 mm2, and matrix size=58×256. T2-weighted images (panels (e) to (f) of FIG. 9) were acquired using a turbo spin-echo sequence and using the following parameters: TR=2900 ms, TE=37 ms, turbo factor=7, FOV=45×45 mm2, slice thickness=1 mm, and matrix size=240×320. The imaged area covered the CCAs, the carotid bifurcation, the internal carotid artery, and the external carotid artery. Contiguous cross-sectional images were obtained perpendicular to the long axis of the neck. Thrombus formation in the CCAs of Example 3 and Comparative Example 2 was observed by MRA and coronal sequential MR images. Before LED irradiation, the vessel lumen in MRA and T2-weighted images is was not significantly different between the CCAs of Example 3 and Comparative Example 2 as illustrated in panels (a) and (e) of FIG. 9. Immediately after intermittent 4.5 mW/cm2 LED irradiation in Example 3, the thrombus appeared on the vessel wall of the CCA of Example 3 as shown in panel (f) of FIG. 9. Because the blood flow of the CCA of Example 3 was disrupted by thrombus, MRA of the CCA of Example 3 was not as bright as the MRA of the CCA of Comparative Example 2 (panel (b) of FIG. 9). Three days after irradiation, as shown in panels (c) and (g) of FIG. 9, the thrombus formed in the CCA of Example 3 was enlarged and significantly disrupted blood flow. Seven days after irradiation, the CCA of Example 3 was totally occluded (panel (h) of FIG. 9), which caused signal disappearance of the CCA of Example 3 on the MRA (panel (d) of FIG. 9).

### [Histological and Immunohistochemical Staining]

The animal of Example 3 and Comparative Example 2 was anesthetized and sacrificed. Both CCAs of Example 3 and Comparative Example 2 were removed and washed in phosphate buffer solution, dehydrated on dry ice, and stored at −80° C. for 24 hours. Before sectioning, all CCAs were embedded in Optimal Cutting Temperature Compound (Tissue Tek, 4583) (Sakura Finetek USA, Inc., Torrance, Calif., USA) and stored at −80° C. for another 12 hours. Cross-sections of the CCAs of Example 3 and Comparative Example 2 (10 μm in thickness) were consecutively cut on a microtome-cryostat. Then, these sections were subjected to hematoxylin and eosin (H&E) staining for evaluation of thrombus formation. For immunohistochemical staining, the slides were treated with cold acetone for 10 minutes and then with methanol with 0.3% H2O2 for 10 minutes. After blocking endogenous peroxidase using a peroxidase blocking reagent (DAKO, Carpinteria, Calif., USA), the primary antibody (anti-PECAM-1 [anti-CD31]) (AbD Serotec, Dusseldorf, Germany) was applied at a dilution of 1:500 overnight at 4° C., followed by a universal immuno-peroxidase polymer (Histofine) (Cosmo Bio, Carlsbad, Calif., USA) for 1 hour at room temperature. Development of color was achieved by exposure for 2 minutes to the DAB chromogen system (DAKO). Endothelium cells were considered positive for PECAM-1 in the presence of intense brown staining.

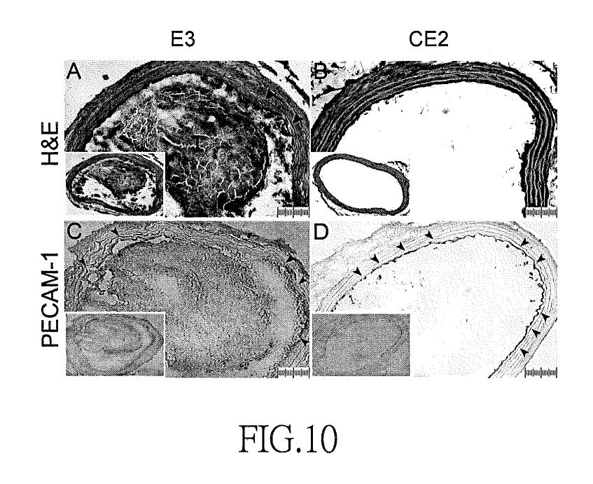

Histologic examination of the CCAs of Example 3 and Comparative Example 2 after intermittent LED irradiation for 3 days in Example 3 are illustrated in FIG. 10, where panel (A) shows that the CCA of Example 3 had formation of thrombus packed with red blood cells, leucocytes, and platelets, whereas no thrombus formation was observed in the CCA of Comparative Example 3 (panel (B)). The endothelium of the CCA of Example 3 was injured and platelet adhesion occurred. In contrast, the CCA wall of Comparative Example 2 remained smooth and thin. PECAM-1 expression in vessels of Example 3 and Comparative Example 2 is shown in panels (C) and (D) of FIG. 10. After intermittent LED irradiation, except for the platelet adhesion, the endothelial cells of Example 3 were also injured and discontinued. However, the endothelial cells in the CCA of Comparative Example 2 were intact.

While the disclosure has been described in connection with what is considered the exemplary embodiments, it is understood that this disclosure is not limited to the disclosed embodiments but is intended to cover various arrangements included within the spirit and scope of the broadest interpretation so as to encompass all such modifications and equivalent arrangements.

1. A method for inducing thrombosis in an artery of a living animal, comprising:
implanting an implantable medical device into a living animal, the implantable medical device including a light-emitting element and a wireless power receiver;
administrating a photo-sensitizing dye material into the living animal; and
providing wirelessly a control signal, from an external power controller, to the wireless power receiver which transmits electrical power to the light-emitting element in response to the control signal to drive the light-emitting element to emit light for irradiating the artery of the living animal.

2. The method of claim 1, wherein the light-emitting element is a light-emitting diode.

3. The method of claim 2, wherein the light-emitting element is driven to irradiate the artery with a light power that ranges from about 4.3 mW/cm² to less than 15 mW/cm².

4. The method of claim 3, wherein the light power is not less than about 6 mW/cm².

5. The method of claim 4, wherein the light-emitting element is driven to irradiate the artery continuously for a time period of about four hours.

6. The method of claim 3, wherein the light power ranges from 4.3 to 4.5 mW/cm².

7. The method of claim 6, wherein the light-emitting element is first driven to irradiate the artery continuously for a first time period, is then shut off after the first time period for a second time period, and is further driven to irradiate the artery continuously after the second time period for a third time period.

8. The method of claim 7, wherein the first time period is about two hours, the second time period is about thirty minutes, and the third time period is about two hours.

9. The method of claim 1, wherein the photo-sensitizing dye material is rose bengal.

10. The method of claim 1, wherein the living animal is a rodent.

11. The method of claim 10, wherein the rodent is a mouse or a rat.

12. A system for inducing thrombosis in an artery of a living animal, said system comprising:
an external power controller operable to wirelessly transmit a control signal; and
an implantable medical device configured to be implanted in the living animal and including a light-emitting element, and a wireless power receiver which is configured to wirelessly receive the control signal from said external power controller, said wireless power receiver being operable to transmit electrical power to said light-emitting element in response to the control signal for driving said light-emitting element to emit light for irradiating the artery of the living animal.

13. The system of claim 12, wherein said external power controller includes a transmitting coil to wirelessly transmit the control signal.

14. The system of claim 12, wherein the control signal has a frequency of about 1 MHz.

15. The system of claim 12, wherein said wireless power receiver is configured as a receiving coil.

16. The system of claim 12, wherein said light-emitting element and said wireless power receiver form parts of an irradiation module that is configured to be secured to the artery of the living animal.

17. The system of claim 12, wherein said implantable medical device further includes an encapsulator that is made of a bio-compatible material and that hermetically encapsulates said irradiation module.

18. The system of claim 17, wherein said bio-compatible material is polydimethylsiloxane.

19. The system of claim 17, wherein said encapsulator is formed with an engaging groove, and said implantable medical device further includes a securing strap that engages said engaging groove and that is configured to secure said irradiation module to the artery.

20. The system of claim 12, wherein said implantable medical device further includes a lens element disposed on said light-emitting element.

21. The system of claim 20, wherein said lens element is formed with a ditch configured to receive the artery.

22. The system of claim 12, wherein said light-emitting element is a light-emitting diode.

23. The system of claim 22, wherein said light-emitting element is configured to irradiate the artery with a light power that ranges from about 4.3 mW/cm² to less than 15 mW/cm².

24. The system of claim 23, wherein the light power is not less than about 6 mW/cm².

25. The system of claim 24, wherein said light-emitting element is driven to irradiate the artery continuously for a time period of about four hours.

26. The system of claim 23, wherein the light power ranges from 4.3 to 4.5 mW/cm².

27. The system of claim 26, wherein said light-emitting element is configured to irradiate the artery continuously for a first time period, is then shut off after the first time period for a second time period, and is further configured to irradiate the artery continuously after the second time period for a third time period.

28. The system of claim 27, wherein the first time period is about two hours, the second time period is about thirty minutes, and the third time period is about two hours.

29. An implantable medical device that is configured to be implanted in a living animal and that is operable to induce thrombosis in an artery of the living animal, said implantable medical device comprising:
an irradiation module configured to be secured to the artery and including a light-emitting element, and a wireless power receiver that is configured to wirelessly receive a control signal from an external power controller and that is operable to transmit electrical power to said light-emitting element in response to the control signal for driving said light-emitting element to emit light for irradiating the artery of the living animal; and
an encapsulator that is made of a bio-compatible material and that hermetically encapsulates said irradiation module.

30. The implantable medical device of claim 29, wherein said wireless power receiver is configured as a receiving coil.

31. The implantable medical device of claim 29, wherein said bio-compatible material is polydimethylsiloxane.

32. The implantable medical device of claim 29, wherein said encapsulator is formed with an engaging groove, and said implantable medical device further comprises a securing strap that engages said engaging groove and that is configured to secure said irradiation module to the artery.

33. The implantable medical device of claim 29, further comprising a lens element disposed on said light-emitting element.

34. The implantable medical device of claim 33, wherein said lens element is formed with a ditch configured to receive the artery.

35. The implantable medical device of claim 29, wherein said light-emitting element is a light-emitting diode.

36. An animal model comprising a living animal subjected to the method of claim 1.

37. An animal model comprising a living animal implanted with said implantable medical device of claim 29.

38. The animal model of claim 37, wherein the living animal is a rodent.

39. The animal model of claim 38, wherein the rodent is a mouse or a rat.
